(19) United States
(12) Patent Application Publication
(10) Pub. No.: US 2012/0115180 A1
(43) Pub. Date: May 10, 2012

(54) USE OF ANIMAL CELLS FOR SCREENING PROBIOTIC BACTERIA STRAINS

(75) Inventors: Kaarina Lähteenmäki, Espoo (FI); Jaana Mättö, Helsinki (FI); Harri Mäkivuokko, Kirkkonummi (FI); Jukka Partanen, Helsinki (FI)

(73) Assignee: SUOMEN PUNAINEN RISTI VERIPALVELU, Helsinki (FI)

(21) Appl. No.: 13/384,521

(22) PCT Filed: Jul. 14, 2010
(86) PCT No.: PCT/FI2010/050593

§ 371 Date: Jan. 17, 2012

(30) Foreign Application Priority Data
Jul. 15, 2009 (FI) 20095792

(51) Int. Cl. C12Q 1/02; C12N 1/20

(52) U.S. Cl. 435/29; 435/252.1

(57) ABSTRACT

The present invention relates to an in vitro and/or ex vivo method of screening probiotic bacterial strains. The present invention also relates to a method of assessing quality of probiotic culture. In addition, the present invention relates to use of animal cells in screening of a probiotic strain. The present invention also relates to use of animal cells in assessing quality of probiotic culture.

### FIELD OF THE INVENTION

The present invention relates to an in vitro and/or ex vivo method of screening probiotic bacterial strains. The present invention also relates to a method of assessing quality of probiotic culture or a culture batch. In addition, the present invention relates to use of animal cells in vitro in screening of probiotic strains. The present invention also relates to use of animal cells in vitro in assessing quality of probiotic culture or a culture batch.

### BACKGROUND OF THE INVENTION

Probiotics are by definition “Live microorganisms which when administered in adequate amounts confer a health effect in the host” (FAO/WHO 2002). Currently, most of the probiotic strains intended for human consumption belong to Lactobacillus or Bifidobacterium genera. The use of probiotic products in various intestinal disease and disturbance states is continuously increasing, and competition in the field prompts isolation of novel, effective probiotic strains. There is a clear demand for more reliable methods for identification of the most promising strains as early as possible. A bacterial strain that is classified as a probiotic must have beneficial effects on intestinal health, but in addition to the actual effector properties, there are several other factors that are crucial for the utilization of the strain. In addition to several safety and other functional criteria, it is important that the strains need to survive in the target tissue e.g. in the intestine. Currently, the tolerance of bile and acidic conditions are properties that are studied while screening probiotic strains (Bezkorovainy A. (2001) Am J Clin Nutr. 73, 399S-405S; Delgado et al. (2007) J Food Sci. 72:M310-5). Production of antimicrobial substances that inhibit pathogenic bacteria is also a desirable metabolic activity for a probiotic strain. Further, one of the methods commonly used for screening of probiotic strains is adhesion to intestinal epithelial cells, as it is thought to be one possibly critical factor required for colonization. Unfortunately, the current tests are not satisfactory: for example, test conditions have a marked influence on the results of the in vitro functionality assays and adhesion assays have shown variable results between in vitro models and between laboratories (Jacobsen et al. (1999) Appl Environ Microbiol 65: 4949-4956; Tuomola et al. (2001) Am J Clin Nutr 73: 393S-398S). Although adhesion to epithelial cells or mucus is considered as one of the basic criteria for probiotic selection, adhesion capabilities of the pro-biotic strains currently used in commercial preparations are highly variable (Jacobsen et al. 1999). Moreover, reports on the human (Jacobsen et al. 1999) and mice intervention (Ibnou-Zekri et al. (2003) Infect Immun. 71: 428-436) trials have shown that the adhesion property did not predict the in vivo colonization capability of a probiotic strain. Indeed, very few of the probiotic strains selected on the basis of the current selection criteria have turned out to be able to colonize the intestinal tract for a significant period of time (Dunne et al. (2001) Am J Clin Nutr. 73, 386S-392S). Although the desired probiotic characteristics have been defined (FAO/WHO, 2002), no standard criteria (such as cut-off values for different criteria) or assays exist for functionality assays. Typically the choice of the potential probiotic strains is based on the selection of the best strains from an intraspecies comparison on a relative basis. In addition to selection of novel probiotic strains, in vitro functionality assays are needed for assessment of quality variation of probiotic cultures or product batches. For example, large differences in the adherence and colonization properties between production lots of the same strain have been reported (Tuomola et al. 2001). Other situations in which quality aspects are tested include long periods of storage or changes in production process, to give but a few examples. Growth properties in relation to the actual target cells in the intestine have not been resolved. Moreover, the dose response of probiotics is poorly known. Novel robust in vitro methods for screening of probiotic strains are required, because the methods currently in use do not yet adequately predict the survival and efficacy of the strains in the intestine.

### BRIEF DESCRIPTION OF THE INVENTION

An object of the present invention is to provide an in vitro and/or ex vivo method of screening and/or isolating probiotic bacterial strains. A further object of the present invention is to provide a specific probiotic strain discovered by the screening and/or isolation method of the invention. Another object of the present invention is to provide a method of estimating a dose of a probiotic needed for a desired effect. A further object of the present invention is to provide a method of assessing quality of a probiotic culture or a culture batch. An additional object of the present invention is to provide an in vitro method of screening variation between growth of a bacterial strain on and/or in the presence of animal cells derived from an individual affected with a disease and growth of the same strain on and/or in the presence of animal cells derived from an individual not affected with the disease. In addition, an object of the present invention is a use of an animal cell in vitro and/or ex vivo in screening and/or isolating of a probiotic strain. A further object of the present invention is a use of animal cells in estimating a dose of a probiotic needed for a desired effect. Further, an object of the present invention is a use of animal cells in assessing quality of probiotic culture or a culture batch. An additional object of the present invention is to provide a use of an animal cell in vitro and/or ex vivo in screening variation between growth of a bacterial strain on and/or in the presence of animal cells derived from an individual affected with a disease and growth of the same strain on and/or in the presence of animal cells derived from an individual not affected with the disease.

The invention is based on the observation that the growth of certain probiotic and/or intestinally derived bacterial strains in vitro is augmented in the presence of human intestinal epithelial cells or fibroblasts, while other strains show only survival without profound growth and some even die. Accordingly, the current invention provides a novel and effective means for screening of potentially probiotic strains and for assessment of quality of probiotic cultures or culture batches.

The objects of the invention are achieved by the methods, products and uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 illustrates the growth of L. rhamnosus GG (VTT E-96666) on HT-29 epithelial cells and in plain cell culture medium. Bacteria were inoculated into the wells, and viable counts were determined after 18 h incubation. The results shown are averages of four samples originating from two independent experiments.

FIG. 2 illustrates the growth of Lactobacillus spp. strains in the presence of HT-29 epithelial cells. The results are shown as the amount of bacteria from HT-29 cell-containing wells compared to the amount of bacteria from plain media-containing wells (averages of duplicate samples).

FIG. 3 illustrates the growth of L. rhamnosus GG (VTT E-96666), L. acidophilus VTT E-96276^{T} (=DSM 20079) and L. casei VTT E-96710^{NT} (=LMG 17314) on Caco-2 colonic epithelial cells and in plain cell culture medium. Bacteria were inoculated into the wells, and viable counts were determined after 18 h incubation. The results shown are averages of triplicate samples.

FIG. 4 illustrates the growth of L. rhamnosus GG (VTT E-96666), L. acidophilus VTT E-96276^{T} (=DSM 20079) and L. casei VTT E-96710^{NT} (=LMG 17314) on HuTu80 small epithelial cells and in plain cell culture medium. Bacteria were inoculated into the wells, and viable counts were determined after 18 h incubation. The results shown are averages of triplicate samples.

FIG. 5 illustrates the growth of L. rhamnosus GG (VTT E-96666) on HGF-1 gingival fibroblast cells and in plain cell culture medium. Bacteria were inoculated into the wells, and viable counts were determined after 18 h incubation. The results shown are averages of triplicate samples.

FIG. 6 illustrates the growth of L. rhamnosus GG (VTT E-96666) and L. casei VTT E-96710^{NT} on HT-29 cells, on HT-29-cell conditioned medium and in plain cell culture medium. Bacteria were inoculated into the wells, and viable counts were determined after 18 h incubation. The results are shown as averages of duplicate samples.

FIG. 7 illustrates the growth of Bifidobacterium type strains in the presence HT-29 epithelial cells. The results are shown as the amount of bacteria from HT-29 cell-containing wells compared to the amount of bacteria from plain media-containing wells. L. rhamnosus GG (VTT E-96666) is included as a control. The results are shown as averages of duplicate samples.

FIG. 8 shows the absorbance measurement (OD A595ₙₘ) after 18 hour co-culture of L. rhamnosus GG (VTT E-96666), L. acidophilus VTT E-96276¹ and Lactobacillus casei VTT E-96710^{NT} with HT-29 cells and in plain cell culture medium.

FIG. 9 shows the absorbance measurement (OD A595ₙₘ) after 24 and 48 hour co-culture of L. rhamnosus GG (VTT E-96666), L. acidophilus VTT E-96276^{T} and Lactobacillus casei VTT E-96710^{NT} with HT-29 cells and in plain cell culture medium.

FIG. 10 illustrates bile tolerance of L. casei VTT E-96710^{NT} (CAS) and L. rhamnosus GG (VTT E-96666; LGG). Bacteria were cultivated in the presence of HT-29 epithelial cells for 24 h (/HT), in MRS broth for 24 h (stationary growth phase; /MRS stat) or in MRS broth for 7 h (logarithmic growth phase; /MRS log). Bacteria were collected, washed twice with PBS and adjusted to the same cell density (1×10⁸ cells/ml). Bacteria were then incubated on 96-well plates with indicated concentrations of Oxgall in MRS broth anaerobically for 24 h. After incubation the density of bacterial suspensions was determined by measuring A595 nm with Multiscan reader. The results are average values of triplicate samples, and standard deviations are indicated by error bars.

### DETAILED DESCRIPTION OF THE INVENTION

Although probiotic strains have beneficial health effect in a host, they may differ from each other in several features, such as their adhesion to intestinal epithelial cells and antimicrobial characteristics. For example, strains, L. rhamnosus GG and L. plantarum 299v, both adhere to intestinal epithelial cells, but their antimicrobial activity against the intestinal normal flora differs.

The invention is based on the finding that in humans the growth of certain known probiotic strains such as L. rhamnosus GG, as well as certain intestinally derived bacterial strains is augmented in the presence of intestinal epithelial cells or fibroblasts in vitro, while other known probiotic strains, such as L. plantarum 299v, as well as certain intestinally derived bacterial strains show only survival without profound growth and some strains even die in the presence of the intestinal epithelial cells and/or fibroblasts. The strains showing a good growth in the presence of a human cell also presented desired properties for probiotics, in particular, tolerance to bile. This observation discovered with human cells applies equally to non-human animal cells, such as cells of domestic animals and poultry.

On the basis of this finding, a method suitable for screening probiotic strains and/or for screening microbes playing a potential role in the pathogenesis of diseases, has been developed. The probiotic strain so discovered has beneficial effects on the health and/or well-being of the host animal and can be formulated into functional food products, nutritional supplements or miocrobial preparations, for example. The probiotic strain so discovered can, in the similar way as in humans, have beneficial effects on the health and/or well-being of a non-human animal as well. The same principle that was shown to work in man, obviously is valid in other animals. Examples of the use of microbial products in non-human animals are well known in the art and include, just to give a few examples, microbial compositions aimed to compete against or inhibit Salmonella infection in domestic birds such as chicken, or preventing diarrhoea in farm animals (see e.g. Sissons J W. Potential of probiotic organisms to prevent diarrhoea and promote digestion in farm animals. Journal of the Science of Food and Agriculture 2006; 49:1-13).

The bacterial strains which grow well on and/or in the presence of animal cells, such as cells of mucosal origin or fibroblasts, have a growth advantage among the numerous and diverse microbiota present in the intestine. This in turn improves the health promoting or maintaining efficacy and/or probiotic efficacy of the strain.

The observed variable growth of the studied Lactobacillus and Bifidobacterium strains in the presence of intestinal epithelial cells or a fibroblast cell line provides an explanation for the significant strain-specific differences in the beneficial effects that lactic acid bacteria have been shown to have. Bacterial adhesion to HT-29 and Caco-2 cells has earlier been considered to be an indication of probiotic potential of a bacterium. These results demonstrate that strain-specific differences also in other aspects of microbe-host cell interaction are critical in selection of health improving or maintaining strains and/or probiotic strains.

The results indicate that among type strains of Lactobacillus and Bifidobacterium as well as among bacterial isolates from human intestine, there are strains with vigorously stimulated growth in the presence of human cells, such as cells of mucosal origin and/or fibroblasts.

In the present invention the term ‘probiotic’ refers to any bacterial species, strain or their combinations, with health promoting, maintaining and/or supporting effects, not limited to strains that are currently accepted as probiotics.

In the present invention, the term ‘a cell of mucosal origin’ refers to a cell found on and/or derived from skin, gastrointestinal tract, in particular the gut, nasopharynx (nose, mouth and ears), vaginal, and/or alveolar tract (the lungs). In one embodiment of the invention, the cell is a mucosal epithelial cell, such as an intestinal epithelial cell. In another embodiment of the invention, the cell is of a gingival origin, for screening probiotics particularly suitable for modulation of oral disorders.

In the present invention, the term ‘an animal’ refers in addition to humans to other mammals such as dogs, cats, horses, pigs and to poultry.

Utilization of this finding in selection of health improving or maintaining strains and/or probiotic strains aids the identification of effective health improving or maintaining strains and/or probiotic strains. Accordingly, the invention relates to a method of screening probiotic strains in vitro and/or ex vivo, comprising growing bacteria on and/or in the presence of animal cells and detecting the extent of the growth of the bacteria. According to one embodiment, the invention relates to a method of screening probiotic strains in vitro and/or ex vivo comprising growing bacteria on and/or in the presence of an animal cell of mucosal origin and detecting the extent of the growth of the bacteria. According to another embodiment, the invention relates to a method of screening probiotic strains in vitro and/or ex vivo comprising growing bacteria on and/or in the presence of a fibroblast and detecting the extent of the growth of the bacteria. The method may also contain additional and/or optional steps that are conventional to methods of growing and/or culturing bacterial cells, such as washing, incubating and dividing the cell populations.

The present invention further relates to a method of isolating a probiotic strain in vitro and/or ex vivo comprising growing bacteria on and/or in the presence of animal cells, detecting the extent of the growth of the bacteria and isolating the desired bacterial strain. According to one embodiment, the invention relates to a method of isolating a probiotic strain in vitro and/or ex vivo comprising growing bacteria on and/or in the presence of an animal cell of mucosal origin and detecting the extent of the growth of the bacteria. According to another embodiment, the invention relates to a method of isolating a probiotic strain in vitro and/or ex vivo comprising growing bacteria on and/or in the presence of a fibroblast and detecting the extent of the growth of the bacteria. The method may also contain additional and/or optional steps that are conventional to methods of growing and/or culturing bacterial cells, such as washing, incubating and dividing the cell populations.

It is also noted that the animal cell may also have other origins than the mucosal tissue or fibroblast, if they still show the same ability to both support the growth of health improving or maintaining bacterial strains and/or probiotics and predict the functionality of the bacteria. Some established cell lines may be more readily cultivated or be more safe in use.

The invention can also be applied in further developments of current probiotics when novel sub strains with better colonisation abilities are screened e.g. using in vitro mutagenesis or other methods. This can be particularly useful when a probiotic with many desirable properties but not sufficient colonisation ability has been identified and novel sub strains from the original one are screened.

Specific health improving or maintaining bacterial strains and/or probiotic strains discovered by the screening and/or isolation method of the invention are further objects of the present invention. In one embodiment of the invention, the growth of the probiotic strain on an animal cell and/or in the presence of an animal cell in vitro and/or ex vivo is higher than the growth without the presence of an animal cell. In a further embodiment of the invention, the probiotic strains belong to Lactobacillus or Bifidobacterium genera.

On the basis of the finding that the growth of a certain intestinally derived bacterial strain is augmented in the presence of animal cells aids also in predicting the effective dose of the strain needed to achieve desired and/or required effect in the host. Thus, the present invention further relates to a method of estimating a dose of a probiotic needed for a desired effect. In one embodiment, the invention is used in estimating the relative amounts of different probiotics added in a mixture of probiotics. It can be assumed that probiotics with good growth as described in the present invention may be needed in smaller relative amounts than those with poor growth.

Further, the finding of the invention can be utilized in assessing quality and/or functionality of probiotic culture or a culture batch. Accordingly, the present invention relates to a method of assessing quality of probiotic culture comprising growing bacteria on and/or in the presence of animal cells in vitro and/or ex vivo and detecting the extent of the growth of the bacteria. According to one embodiment, the invention relates to a method of assessing quality of probiotic culture comprising growing bacteria on and/or in the presence of an animal cell of mucosal origin in vitro and/or ex vivo and detecting the extent of the growth of the bacteria. According to another embodiment, the invention relates to a method of assessing quality of probiotic culture comprising growing bacteria on and/or in the presence of a fibroblast and/or ex vivo and detecting the extent of the growth of the bacteria. The method may also contain additional and/or optional steps that are conventional to methods of assessing quality of cultures, such as washing, incubating, dividing the cell populations and/or comparing the growth within production lots, for example. Situations in which quality aspects, such as lot-to-lot variation in the functionality, are tested include a long period of storage and/or changes in production process, for example. It is of note that the cell or cell line may not necessarily have the same host animal origin but can as well be of any origin, once the relevant functionality of the cell has been demonstrated. For example, in quality control, it could be reasonable and/or more economical to use a non-human cell or cell line instead of a human cell or cell line.

The present invention further relates to a method of screening microbe strains whose ability of growth on animal cells in vitro differs between affected and non-affected tissues or cells. The term “affected” here refers to any disease which is related to gut microbiota, such as, type I diabetes, allergy, celiac disease and inflammatory bowel disease (IBD) and similarly, the terms “affected cells” or “affected tissues” refer to those isolated from individuals with the disease. Thus, the present invention relates to a method of assessing variation in growth of a bacterial strain on and/or in the presence of animal cells in vitro and/or ex vivo derived from an individual affected with a disease and growth of the same strain on and/or in the presence of animal cells derived from an individual not affected with the disease. In other words, the present invention relates to a method of screening growth of a bacterial strain on and/or in the presence of human cells in vitro and/or ex vivo derived from an individual affected with a disease and growth of the same strain on and/or in the presence of animal cells derived from an individual not affected with the disease. As the invention provides means for identification of microbes that have different abilities to grow in the presence of affected versus healthy tissue, microbes playing a potential role in the pathogenesis of diseases can be identified.

In one embodiment of the invention, the animal cell is a cell of mucosal origin, such as an intestinal epithelial cell. In another embodiment, the animal cell is a fibroblast.

In addition, the present invention relates to in vitro use of an animal cell in screening of health improving or maintaining bacterial strains or probiotic strains. The present invention also relates to use of an animal cell in isolating a probiotic strain. The present invention further relates to use of an animal cell in estimating a dose of a probiotic needed for a desired effect. Further, the present invention relates to use of an animal cell in assessing quality of probiotic culture and/or a culture batch.

In addition, the present invention relates to use of an animal cell in screening variation between growth of a bacterial strain on and/or in the presence of animal cells derived from an individual affected with a disease and growth of the same strain on and/or in the presence of animal cells derived from an individual not affected with the disease. Thus, the present invention relates to a use of an animal cell in screening microbe strains whose in vitro ability of growth on mucosal tissues or cells differ between affected and non-affected individuals.

In one embodiment of the invention, the animal cell is a cell of mucosal origin. In another embodiment, the animal cell is a fibroblast. In a further embodiment of the invention, the cell is an epithelial cell of mucosal origin, such as an intestinal epithelial cell. In another further embodiment of the invention, the animal cell is a human cell. In a further embodiment the cell is selected from HT-29 cells, Caco-2 cells, HGF-1 cells and/or HuTu80 cells.

In one embodiment of the invention, the probiotic bacteria belong to Lactobacillus or Bifidobacterium genera.

The invention will be described in more detail by means of the following examples. The examples are not to be construed to limit the claims in any manner whatsoever.

### EXAMPLES

### Materials and Methods

The materials and methods describes herein are common to examples 1 to 8.

### Cultivation of Eukaryotic Cells

Eukaryotic cells were cultivated to semiconfluency and divided onto 48-well plates at a concentration of 1×10⁶ cells/ml (HT-29, Caco-2, HuTu80) or 1×10⁶ cell/ml (HGF-1) in complete cell culture medium supplemented with fetal bovine serum (FBS, Gibco). The same number of wells obtained an equal amount of cell culture medium without the cells. The plates were incubated at +37° C., 5% CO₂ atmosphere o/n, after which the medium was removed, the wells were washed twice with phosphate buffered saline, pH 7.2 (PBS), and fresh, serum-free medium was added on the wells.

### Cultivation and Enumeration of Bacteria

The bacteria were cultivated anaerobically in MRS-broth (medium formulation by by deMan, Rogosa and Sharpe; Lactobacillus strains) or in RCM-broth (Robertson's cooked meat broth) supplemented with 0.5 g/l L-cysteine-monohydrate (Bifidobacterium strains) at +37° C. o/n, collected by centrifugation and washed twice with PBS. The bacteria were inoculated into the wells containing the culture medium and epithelial cells, or into the wells containing plain culture medium, at a concentration of ca. 1×10⁵ bacteria/ml. The plates were incubated at +37° C., 5% CO₂ atmosphere for 18 h. After incubation, numbers of viable bacteria in the wells was determined. To ensure that also the bacteria that may have adhered on the epithelial cell surface were liberated into the medium, the wells were treated for 45 min with 0.2% Triton-X-100 (in H₂O) which lyses the eukaryotic cells. After this, serial dilutions of the media from the wells were plated on MRS- or RCM-agar plates for determination of viable counts. Bacterial number in the wells containing epithelial cells was compared to the number of bacteria in plain cell culture medium and to the inoculum.

### Example 1

Effect of HT-29 Colonic Intestinal Epithelial Cells on the Growth of Lactobacillus rhamnosus GG

HT-29 colonic intestinal epithelial cells (ATCC HTB-38) were cultivated in McCoy's 5A medium (Gibco) supplemented with 10% FBS. The probiotic strain Lactobacillus rhamnosus GG (VTT E-96666, strain GG) was inoculated onto HT-29 cells in serum-free McCoy's 5A medium, as well as in plain serum-free McCoy's 5A medium. After 18 h incubation, the number of viable bacteria in the wells was determined as described above.

The results are shown in FIG. 1. As shown in FIG. 1, L. rhamnosus GG grew to more than 100-fold numbers in the presence of HT-29 cells. On the contrary, the bacteria incubated in plain cell culture medium showed only modest growth.

### Example 2

Effect of HT-29 Epithelial Cells on the Growth of Various Lactobacillus spp. Strains

It was studied whether the epithelial cell-mediated stimulation of growth is a common property among lactic acid bacteria. The growth stimulation experiments were performed as described in the example 1.

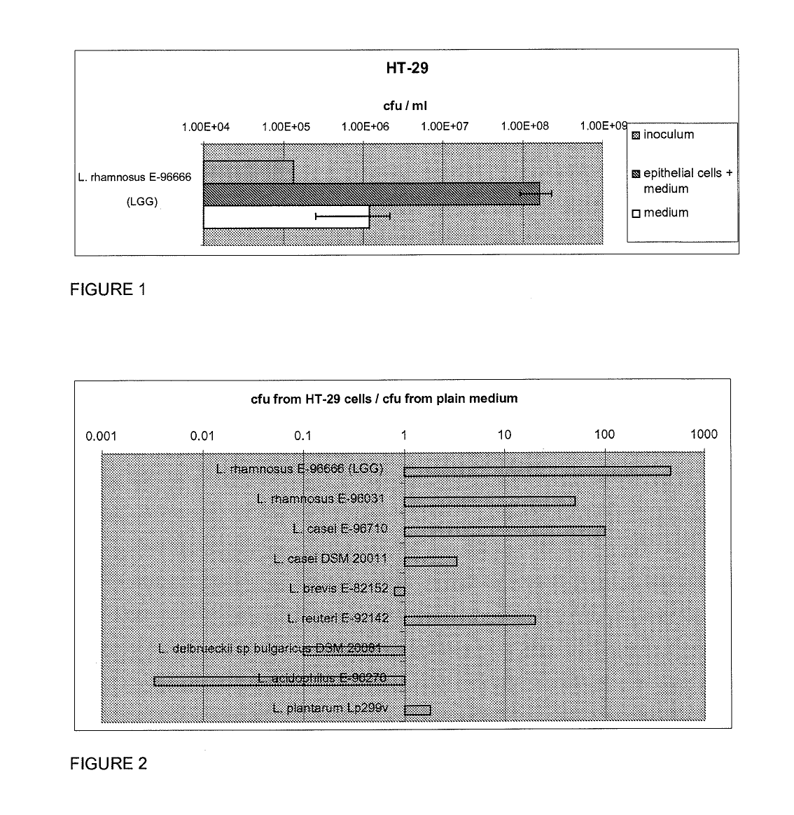

The results are shown in FIG. 2. The results with 9 Lactobacillus strains L. rhamnosus GG (VTT E-96666), L. rhamnosus VTT E-96031^{T}, L. casei VTT E-96710^{NT}, L. casei DSM 20011^{T}, L. brevis VTT E-82152 (=ATCC 367), L. reuteri VTT E-92142^{T}, L. delbrueckii subsp. bulgaricus DSM 20081^{T}, L. acidophilus VTT E-96276^{T} and, and L. plantarum Lp299v show that there are strains whose growth is efficiently stimulated by HT-29 cells (results are shown as the amount of bacteria from HT-29 cell-containing wells compared to the amount of bacteria from plain media-containing wells). However, strains whose growth was not stimulated by the HT-29 epithelial cells were also found and some strains even died more rapidly in the presence of the HT-29 epithelial cells than in plain cell culture medium were found. Intraspecies differences in the two distinct strains representing L. rhamnosus and L. casei species were also observed. These results show that Lactobacillus spp. includes species and strains with highly differing ability to survive in the intestinal epithelium and that the stimulation effect of the epithelial cells is strain-specific.

### Example 3

### Effect of Caco-2 Colonic Intestinal Epithelial Cells on the Growth of Lactic Acid Bacteria

The effect of Caco-2 intestinal epithelial cells (ATCC HTB-37) on the growth of L. rhamnosus GG (VTT E-96666), L. acidophilus VTT E-96276^{T} (=DSM 20079) and L. casei VTT E-96710^{NT} (=LMG 17314) was measured. The Caco-2 cells were cultivated in Minimal essential medium (MEM) (Gibco) supplemented with L-glutamine (Gibco), sodium pyruvate (Gibco) and 20% FBS. Bacterial survival was measured in serum-free culture medium as described above.

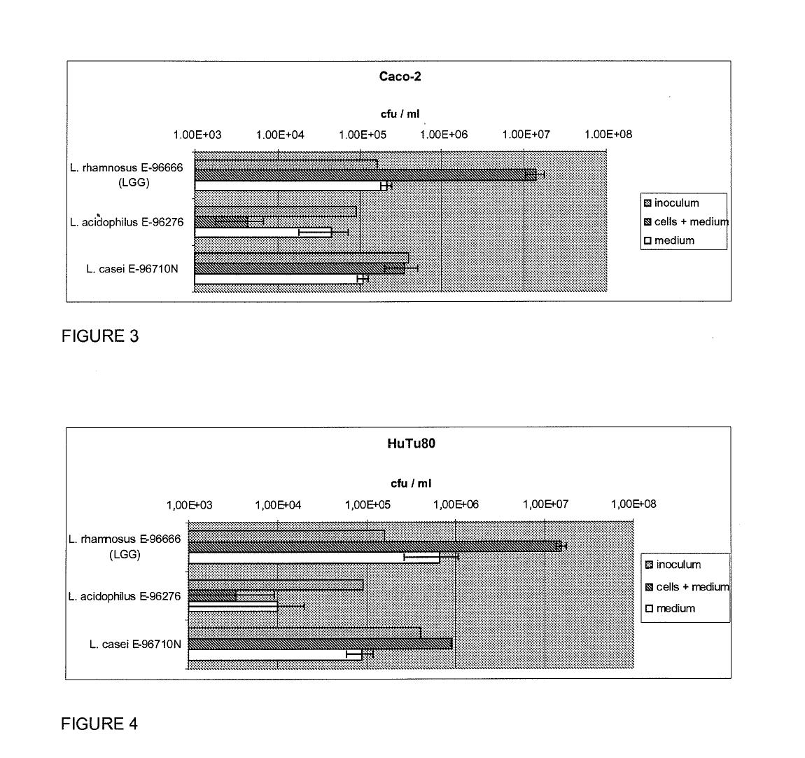

The results are shown in FIG. 3. As shown in FIG. 3, the number of L. rhamnosus GG cells, a common probiotic strain, increased a 100-fold in the presence of Caco-2 cells, whereas only a minimal increase was seen in the absence of Caco-2 cells. Growth stimulation by Caco-2 epithelial cells was not observed in the other two Lactobacillus sp. type strains. These results demonstrate that Caco-2 colonic intestinal epithelial cells stimulate the growth of LGG and the growth stimulation characteristic is variable in lactobacilli.

### Example 4

### Effect of Small Intestinal Epithelial Cells (HuTu80) on the Growth of Lactic Acid Bacteria

The growth of L. rhamnosus GG (VTT E-96666), L. acidophilus VTT E-96276^{T} (=DSM 20079) and L. casei VTT E-96710^{NT} (=LMG 17314) in the presence of HuTu80 epithelial cells (ATCC HTB-40) that are derived from small intestine was studied. HuTu80 cells were cultivated in Minimal essential medium (MEM) (Gibco) supplemented with L-glutamine (Gibco), sodium pyruvate (Gibco) and 10% FBS. Bacterial survival was measured in serum-free culture medium as described above.

The results are shown in FIG. 4. As shown in FIG. 4, the number of L. rhamnosus GG cells increased a 100-fold in the presence of HuTu80 cells, whereas only a minimal increase was seen in the absence of HuTu80 cells. Growth stimulation by HuTu80 epithelial cells was not observed in the other two Lactobacillus sp. strains. These results demonstrate that also small intestinal epithelial cells stimulate the growth of L. rhamnosus GG and the growth stimulation characteristic is variable in lactobacilli.

### Example 5

### Effect of Gingival Fibroblast Cells (HGF-1) on the Growth of Lactic Acid Bacteria

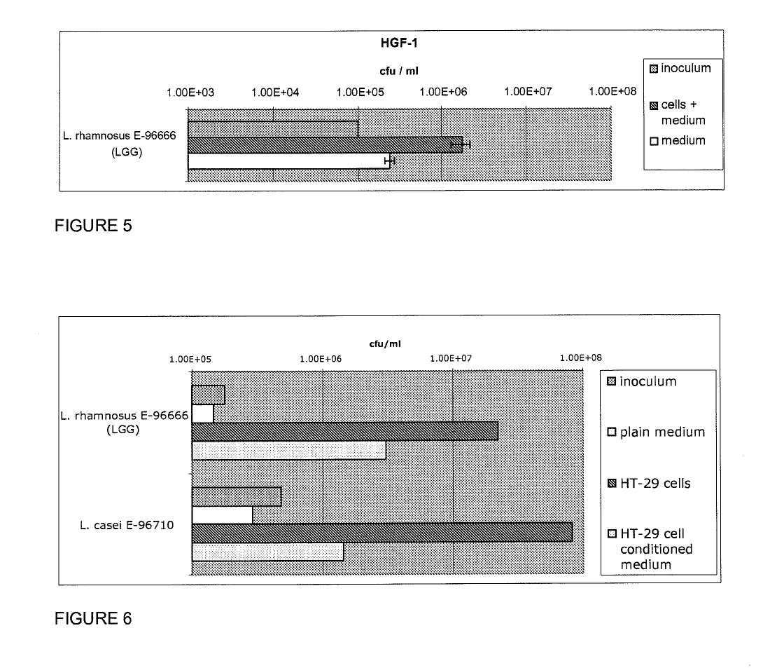

The growth of L. rhamnosus GG (VTT E-96666) in the presence of HGF-1 fibroblast cells (ATCC CRL-2014) that are derived from the human gingiva was studied. HGF-1 cells were cultivated in Dulbecco's Modified Eagles's Medium (ATCC) supplemented with 10% FBS (10⁵ cells/ml density). Bacterial survival was measured in serum-free culture medium as described above. The results are shown in FIG. 5. As shown in FIG. 5, the number of L. rhamnosus GG increased approximately ten-fold in the presence of HGF-1 cells.

### Example 6

HT-29 Epithelial Cells Stimulate Bacterial Growth More Efficiently than the Medium Preconditioned with HT-29 Cells

To find out whether the screening method could utilize bacterial in-oculation on epithelial cell-conditioned medium instead of epithelial cells themselves HT-29 epithelial cells were first incubated with serum-free McCoy's 5A medium for 18 h. The cell-conditioned medium was collected, and the bacteria (L. rhamnosus VTT E-96666 and L. casei VTT E-96710^{NT}) were inoculated in either the cell-conditioned medium, on HT-29 cells, or in plain cell culture medium. After 18 h incubation, bacterial viable counts were determined.

The results are shown in FIG. 6. As demonstrated in FIG. 6, also the conditioned medium stimulated the growth of bacteria to a moderate extent (20 times [L. rhamnosus GG] and 5 times [L. casei] more bacteria than in plain cell culture medium), however, bacterial numbers in the presence of cells were considerably larger (over 100 times more bacteria than in plain medium).

### Example 7

Effect of HT-29 Epithelial Cells on the Growth of Various Bifidobacterium spp. Strains

It was also studied whether the HT-29 epithelial cell-mediated stimulation can be extended to Bifidobacterium spp. strains. The growth stimulation experiments were performed as described in the example 1.

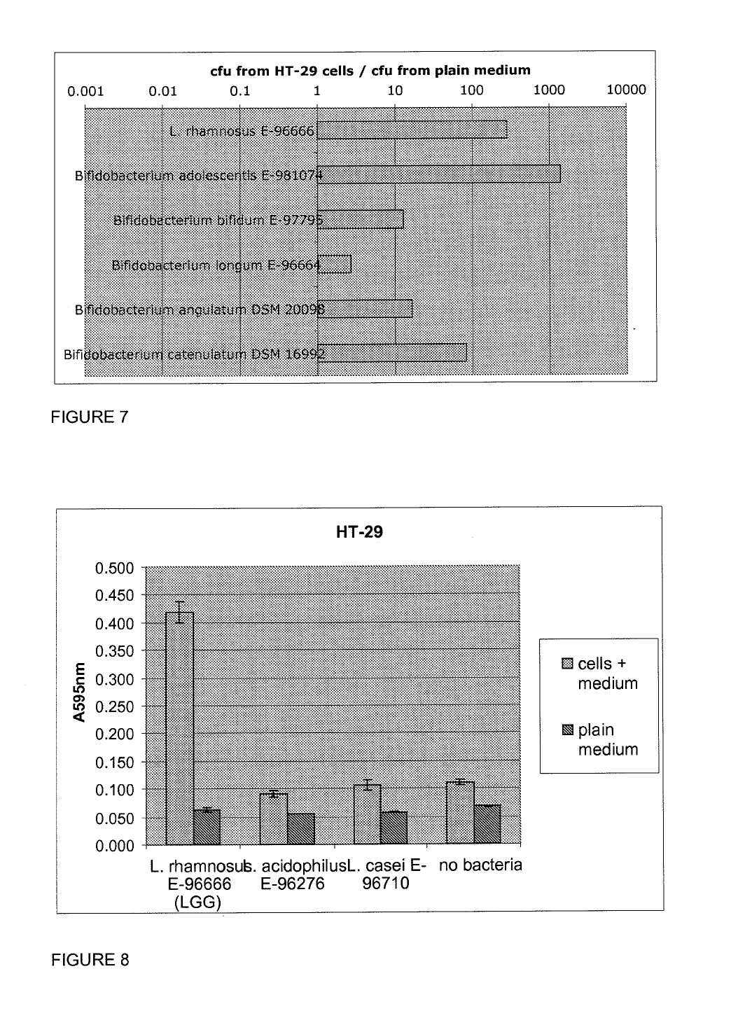

The results are shown in FIG. 7. The results with Bifidobacterium spp. strains B. adolescentis VTT E-981074^{T}, B. bifidum VTT E-97795^{T}, B. longum VTT E-96664^{T}, B. angulatum DSM 20098^{T}, and B. catenulatum DSM 16992^{T} show that there are strains whose growth was efficiently stimulated by HT-29 cells. However, as in the case of Lactobacillus spp., we also found strains whose growth was not stimulated by the epithelial cells.

### Example 8

### Effect of HT-29 Epithelial Cells on the Growth of Various Intestinal Bacterial Isolates

We characterized 44 intestinal bacterial isolates in relation to their growth on HT-29 intestinal epithelial cells. The growth stimulation experiments were performed as described in the example 1. Among the 44 isolates tested, HT-29 cells stimulated the growth of 12 strains 100-fold or more as compared to plain medium, while the growth of 8 strains was inhibited by the HT-29 cells. These results indicate that the method is applicable for comparison of the characteristics of intestinal bacterial strains.

### Example 9

### Absorbance Measurement as a Rapid Detection Method for Bacterial Growth on the Epithelial Cells

In addition to viable culture we measured the optical density (A595 nm) of cell cultures after incubation with the L. rhamnosus GG (VTT E-96666), L. acidophilus VTT E-96276^{T} (=DSM 20079) and L. casei VTT E-96710^{NT} (=LMG 17314) strains. Here, the epithelial cells were cultivated on 96-well plates. After washing the cells, serum-free medium and bacterial inoculum was added as described in the material and methods. The bacteria were inoculated similarly in plain cell culture medium, and the A595ₙₘ was measured after 18 h, 24 h and 48 h incubation.

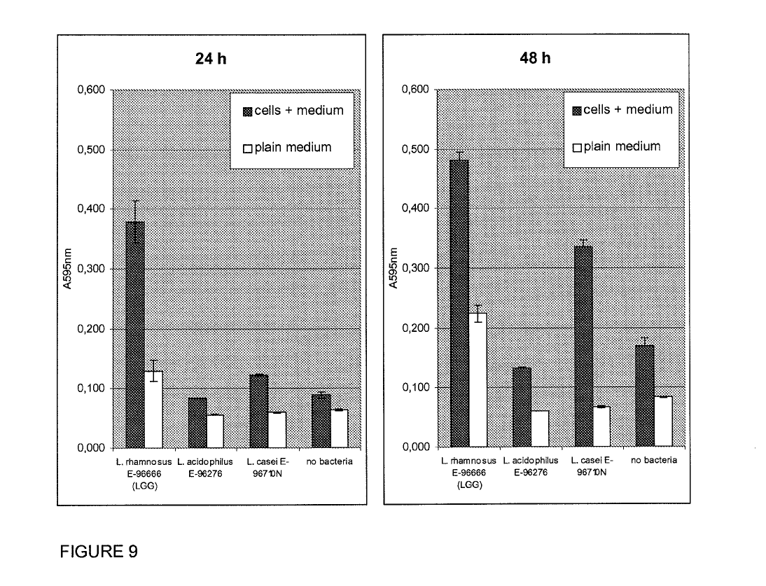

The results are shown in FIGS. 8 and 9. As shown in the FIG. 8, L. rhamnosus GG strain displayed a marked increase in the OD during 18 h co-culture with HT-29 epithelial cells. As shown in the FIG. 9, also L. casei VTT E-96710^{NT} promoted a significant increase in the OD when it was co-cultured with the HT-29 cells for 48 h, while no marked OD increase was observed with L. acidophilus VTT E-96276^{T} strain even in longer incubation with the epithelial cells. The OD in wells containing bacteria in plain culture medium remained significantly lower than in wells containing HT-29 cells. OD measurement can thus be used as a rapid method for the screening of strains with the most prominent epithelial cell growth capability.

### Example 10

### Tolerance of Bile

L. casei VTT E-96710^{NT} (=LMG 17314=ATCC 334) was inoculated in 1/200 dilution and cultivated in the presence of HT-29 epithelial cells for 24 h or in MRS broth for 24 h (stationary growth phase) or in MRS broth for 7 h (logarithmic growth phase). The commonly used probiotic strain L. rhamnosus GG (VTT E-96666), which is known to be highly tolerant to bile, was cultivated similarly. After cultivation, bacteria were collected, washed twice with PBS and adjusted to the same cell density with each other (1×10⁸ cells/ml). Bacteria were then incubated anaerobically on 96-well plates (Nunc) with varying concentrations of Oxgall (dehydrated fresh bovine bile; Difco) in MRS broth for 24 h. After incubation the optical density of bacterial suspensions was determined by measuring A595 nm with Multiscan RC reader (Labsystems).

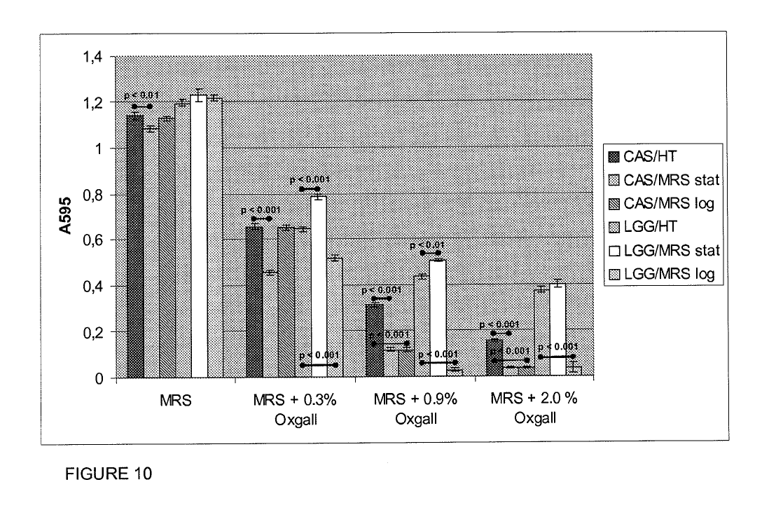

The results are shown in FIG. 10. After cultivation on HT-29 cells, L. casei ATCC334 tolerated even the high 2% concentration of Oxgall, whereas L. casei cultivated under standard laboratory conditions, i.e, in MRS broth to stationary growth phase, was unable to grow in the presence of 2% Oxgall. L. casei bacteria cultivated to stationary phase in MRS broth showed reduced growth already in the presence of 0.3% Oxgall, which was well tolerated by the bacteria cultivated in the presence of epithelial cells. In essence, epithelial cell cultivation increased the bile tolerance of L. casei closer to the level seen with stationary phase L. rhamnosus GG (FIG. 10). Thus, screening according to the present invention revealed strains with properties desired for probiotics.

1-23. (canceled)

24. A method of screening a probiotic strain comprising growing bacteria on and/or in the presence of an animal cell in vitro and/or ex vivo and detecting the extent of the growth of the bacteria.

25. A method of isolating a probiotic strain comprising screening bacteria according to the method of claim 24, selecting the bacterial strains growing well and isolating them.

26. A method of assessing quality of probiotic culture comprising growing bacteria on and/or in the presence of an animal cell in vitro and/or ex vivo and detecting the extent of the growth of the bacteria.

27. A method of estimating a dose of a probiotic needed for a desired effect comprising growing bacteria on and/or in the presence of an animal cell in vitro and/or ex vivo and detecting the extent of the growth of the bacteria.

28. The method as claimed in claim 24, wherein the animal cell is a cell of mucosal origin.

29. The method as claimed in claim 24, wherein the animal cell is a fibroblast.

30. The method as claimed in claim 24, wherein the animal cell is a human cell.

31. The method as claimed in claim 30, wherein the cell is selected from HT-29 cells, Caco-2 cells, HGF-1, and/or HuTu80 cells.

32. The method as claimed in claim 24, wherein the bacteria belong to Lactobacillus or Bifidobacterium genera.

33. A probiotic strain discovered by the method of claim 24, wherein the growth of probiotic strain on and/or in the presence of an animal cell in vitro and/or ex vivo is higher than the growth without the presence of an animal cell.

34. The probiotic strain as claimed in claim 33, wherein the animal cell is a human cell.

35. A method of screening variation between growth of a bacterial strain on and/or in the presence of an animal cell in vitro and/or ex vivo derived from an individual affected with a disease and growth of the same strain on and/or in the presence of an animal cell derived from an individual not affected with the disease.

36. The method as claimed in claim 35, wherein the animal cell is a human cell.

37. The method as claimed in claim 26, wherein the animal cell is a cell of mucosal origin.

38. The method as claimed in claim 26, wherein the animal cell is a fibroblast.

39. The method as claimed in claim 26, wherein the animal cell is a human cell.

40. The method as claimed in claim 27, wherein the animal cell is a cell of mucosal origin.

41. The method as claimed in claim 27, wherein the animal cell is a fibroblast.

42. The method as claimed in claim 27, wherein the animal cell is a human cell.

43. The method as claimed in claim 39, wherein the cell is selected from HT-29 cells, Caco-2 cells, HGF-1, and/or HuTu80 cells.

44. The method as claimed in claim 42, wherein the cell is selected from HT-29 cells, Caco-2 cells, HGF-1, and/or HuTu80 cells.

45. The method as claimed in claim 25, wherein the bacteria belong to Lactobacillus or Bifidobacterium genera.

46. The method as claimed in claim 26, wherein the bacteria belong to Lactobacillus or Bifidobacterium genera.

47. The method as claimed in claim 27, wherein the bacteria belong to Lactobacillus or Bifidobacterium genera.

48. A probiotic strain discovered by the method of claim 25, wherein the growth of probiotic strain on and/or in the presence of an animal cell in vitro and/or ex vivo is higher than the growth without the presence of an animal cell.
